(19) United States
(12) Patent Application Publication
(10) Pub. No.: US 2025/0262198 A1
(43) Pub. Date: Aug. 21, 2025

(54) METHOD FOR TREATING MULTIPLE SCLEROSIS

(71) Applicant: BEIJING INNOCARE PHARMA TECH CO., LTD., Beijing (CN)

(72) Inventors: Jisong CUI, Beijing (CN); Renbin ZHAO, Beijing (CN); Carrie ZHOU, Shanghai (CN); Ruiqin LIU, Beijing (CN); Bin ZHANG, Beijing (CN)

(21) Appl. No.: 19/203,810

(22) Filed: May 9, 2025

Related U.S. Application Data
(63) Continuation of application No. PCT/CN2023/131279, filed on Nov. 13, 2023.
(60) Provisional application No. 63/384,274, filed on Nov. 18, 2022.

(51) Int. Cl. A61K 31/4545; A61K 9/00; A61P 37/00

(52) U.S. Cl. CPC A61K 31/4545; A61K 9/0053; A61P 37/00

(57) ABSTRACT

The present invention is directed to a method for treating multiple sclerosis by orally administering to a subject in need thereof at least 20 mg/day, preferably 50-100 mg/day of orelabrutinib. The method is particularly effective to treat relapsing-remitting multiple sclerosis.

### CROSS-REFERENCE TO RELATED APPLICATION(S)

This application is a continuation of PCT/CN 2023/131279, filed Nov. 13, 2023; which claims the benefit of U.S. Provisional Application No. 63/384,274, filed Nov. 18, 2022. The contents of the above-identified applications are incorporated herein by reference in their entireties.

### FIELD OF THE INVENTION

The present invention relates to a method for treating multiple sclerosis by orally administering to a subject in need thereof 20-100 mg/day of orelabrutinib.

### BACKGROUND OF THE INVENTION

Multiple sclerosis (MS) is a chronic, inflammatory, demyelinating and neurodegenerative disease of the central nervous system (CNS). MS affects 2.1 million individuals worldwide every year, nearly 1 million in US, and is also considered a major cause of economic health burden around the world. Both genetics and environmental factors play important roles in the pathogenesis of MS by activating the immune response and causing inflammation. Patients with MS may have different clinical courses. In most patients, the disease presents as relapsing-remitting MS (RRMS). It is the most prevalent form of MS affecting 85% of individuals.

Multiple sclerosis (MS) is an autoimmune disease with the autoimmune activity directed against central nervous system (CNS) antigens. MS is an inflammatory demyelinating disease of the CNS, characterized clinically by relapses and remissions, often leading to progressive physical impairment. The cause of MS is unknown; however, pathologic, genetic, and immunologic features have been identified which suggest that the disease has an autoimmune basis. Although the antigenic target in MS is believed to be confined within the CNS, a systemic immunoregulatory defect may be present. T cells that were reactive to myelin basic protein (MBP) were detected in the blood of MS patients. Circulating blood cells of MS patients were also primed for enhanced cytokine synthesis. Exaggerated mitogen-inducible cytokine synthesis by peripheral monocytes was measured during the weeks immediately preceding the onset of episodes of relapsing MS. Thus, the exaggerated production of tumor necrosis factor (TNF), interleukin-1 (IL-1), and interferon-γ by circulating blood cells may serve as a peripheral trigger or marker for the induction of demyelinating inflammation in the CNS.

The activity and interactions of B cells, T cells, and myeloid cells are involved in the immunopathological characteristics of multiple sclerosis. Antigen-activated B cells exert effect or functions through antigen presentation and the production of cytokines and antibodies. Macrophages and microglia that are abundant in multiple sclerosis lesions also contribute to tissue damage.

Currently there is no cure for MS, but the course of the disease can be altered favorably with disease-modifying therapy with varying levels of efficacy, and distinct safety and tolerability profiles.

There are still significant unmet medical needs for treating MS to decrease the frequency of relapses and improve radiological lesions.

### BRIEF DESCRIPTION OF THE DRAWINGS

FIG. 1A is a chart showing clinical score of EAE mice treated with orelabrutinib at a dose of 30 mg/kg orally (PO) once per day (QD) or vehicle PO, QD at the indicated days. FIG. 1B is a chart showing the relative change of body weight as a percentage in mice treated with orelabrutinib at a dose of 30 mg/kg PO, QD, or vehicle PO, QD at the indicated days.

FIG. 2 shows the study design of clinical trial (Example 2) to evaluate the safety and efficacy of orally-administered orelabrutinib for treating multiple sclerosis patients.

FIG. 3 shows adjusted mean cumulative number of new Gd+T1 brain lesions by visit up to Week 24 (past-hoc analysis, N=115).

### DETAILED DESCRIPTION OF THE INVENTION

The inventors have discovered that orelabrutinib at a dosage of at least 20 mg/day, and preferably 50-100 mg/day, is effective in treating multiple sclerosis.

Orelabrutinib is a small molecule that was chemically synthesized. The chemical name is 2-(4-phenoxyphenyl)-6-[1-(prop-2-enoyl) piperidin-4-yl]pyridine-3-carboxamide, with a molecular weight of 427.50. The CAS No. is 1655504-04-3. The chemical structure of orelabrutinib is shown below.

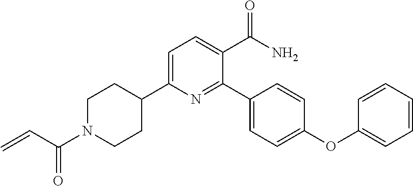 [SMILES: C=CC(=O)N1CCC(c2ccc(C(N)=O)c(-c3ccc(Oc4ccccc4)cc3)n2)CC1]

The present invention provides methods and pharmaceutical compositions for treating a subject having multiple sclerosis and for preserving and/or increasing myelin content in a subject having multiple sclerosis. The method comprises orally administering orelabrutinib at 50-100 mg/day to a subject suffering from multiple sclerosis.

There are four major clinical types of MS that the present invention is useful to treat: 1) relapsing-remitting MS (“RR-MS”), characterized by clearly defined relapses with full recovery or with sequelae and residual deficit upon recovery; periods between disease relapses characterized by a lack of disease progression; 2) secondary progressive MS (“SP-MS”), characterized by initial relapsing remitting course followed by progression with or without occasional relapses, minor remissions, and plateaus; 3) primary progressive MS (“PP-MS”), characterized by disease progression from onset with occasional plateaus and temporary minor improvements allowed; and 4) progressive relapsing MS (“PR-MS”), characterized by progressive disease onset, with clear acute relapses, with or without full recovery; periods between relapses characterized by continuing progression.

In one embodiment, orelabrutinib slows or prevents neurodegeneration including demyelination and neuronal death.

### Pharmaceutical Compositions

The present invention provides pharmaceutical compositions comprising one or more pharmaceutically acceptable carriers and an active compound of orelabrutinib. The active compound in the pharmaceutical compositions in general is in an amount of about 0.1-5% for an injectable formulation, about 1-90% for a tablet formulation, and 1-100% for a capsule formulation.

In one embodiment, the pharmaceutical composition can be in a dosage form such as tablets, capsules, granules, fine granules, powders, syrups, suppositories, injectable solutions, patches, or the like. For example, the active compound can be prepared in Ora-Plus® (an aqueous-based oral suspending vehicle including water, microcrystalline cellulose, carboxymethycellulose, xanthan gum, carrageenan, calcium sulfate, and trisodium phosphate). The above pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, and gels. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as microcrystalline cellulose, methylcellulose, hydroxypropyl cellulose, hydroxyethyl cellulose, carboxymethyl cellulose, and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate; xanthan gum, and carrageenan. Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the active compound may contain other excipients that have no bioactivity and no reaction with the active compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of excipients of a tablet or a capsule include, but not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone. For example, a tablet formulation may contain inactive ingredients such as colloidal silicon dioxide, crospovidone, hypromellose, magnesium stearate, microcrystalline cellulose, polyethylene glycol, sodium starch glycolate, and/or titanium dioxide. A capsule formulation may contain inactive ingredients such as gelatin, magnesium stearate, and/or titanium dioxide.

### Method of Use

The present invention is directed to a method of treating multiple sclerosis. Orelabrutinib can be administered in the form of a pharmaceutical composition that additionally contains a pharmaceutically acceptable carrier. The method comprises the steps of first identifying a subject suffering from multiple sclerosis, and administering to the subject an effective amount of orelabrutinib to treat multiple sclerosis. “An effective amount,” as used herein, is the amount effective to treat a disease by ameliorating the pathological condition or reducing the symptoms of the disease.

In one embodiment, the method treats relapsing-remitting MS, secondary progressive MS, primary progressive MS, and progressive relapsing MS.

In one embodiment, the method comprises the step of orally administering to a subject in need thereof about 50-100 mg/day of orelabrutinib for at least a month. “About” as used herein, refers to ±5% of the recited value.

In one embodiment, the subject is treated with about 50-80 mg/day of orelabrutinib.

In one embodiment, the subject is treated with about 50-80 mg of orelabrutinib once daily.

In one embodiment, the subject is treated with about 80 mg of orelabrutinib once daily.

In one embodiment, the subject is treated for 3 months to 2 years.

In one embodiment, the subject has multiple sclerosis, such as relapsing-remitting multiple sclerosis, and is administered an effective amount of Orelabrutinib for a period of time sufficient to achieve one or more of the following changes: (a) reduced frequency of relapse in the subject, (b) reduced probability of relapse in the subject, (c) reduced annualized relapse rate in the subject, (d) reduced risk of disability progression in the subject, (e) reduced number of new or newly enlarging T2 lesions in the subject, (f) reduced number of new non-enhancing T1 hypointense lesions in the subject, and (g) reduced number of gadolinium (Gd+) lesions in the subject, wherein the changes (a)-(g) are relative to a subject treated with placebo.

In another embodiment, a subject having relapsing-remitting multiple sclerosis is administered an effective amount of Orelabrutinib for a period of time sufficient to achieve one or more of the following changes: (a) reduced annualized relapse rate of at least 30%; (b) reduced risk of disability progression of at least 30%; and (c) reduced number of new or newly enlarging T2 lesions of at least 65% in the subject, wherein the changes (a)-(c) are relative to a subject treated with placebo.

The present invention is also directed to orelabrutinib for use in a method of treating MS, wherein it is orally administered in a dosage of 50-100 mg/day for at least 1 month to a subject in need thereof.

The present invention is further directed to use orelabrutinib in an amount of 50-100 mg for preparing a medicament for treating multiple sclerosis (MS), wherein it is orally administered in a dosage of 50-100 mg/day for at least 1 month to a subject in need thereof.

The pharmaceutical composition of the present invention can be applied by local administration and systemic administration. Local administration includes topical administration. Systemic administration includes oral, parenteral (such as oral, intravenous, intramuscular, subcutaneous or rectal), and other systemic routes of administration. In systemic administration, the active compound first reaches plasma and then distributes into target tissues. Oral administration is a preferred route of administration for the present invention.

Dosing of the composition can vary based on the extent of the injury and each patient's individual response, and the possibility of co-usage with other therapeutic treatments including use of other therapeutic agents.

In general, an effective dose of orelabrutinib to be orally administered to a human subject is about 50-100 mg per day. The daily dosage may be administered in one administration or in separate administrations of 2, 3, 4, or 6 equal doses. For example, the dosage is about 50 mg once a day, or about 50 mg twice a day, or about 80 mg once a day.

In one embodiment, the pharmaceutical composition is administrated subcutaneously to the subject.

The time period for which the subject is dosed with orelabrutinib in any of the methods described above can range, for example, from about 1 week to the remaining lifespan of the subject. Orelabrutinib can be dosed, for example, for equal to or more than 1, 2, 3, 4, 5, 6, 7, 8, 9, 10, 12, 20, 30, 40, or 50 weeks, or for equal to or more than 1, 2, 3, 4, 5, 6, 7, 8, 9, 11, or 12 months, or for equal to or more than 1, 2, 3, 4, 5, 6, 7, 8, 9, 10, 20, 30, 40, or 50 years.

Those of skill in the art will recognize that a wide variety of delivery mechanisms are also suitable for the present invention.

The present invention is useful in treating a mammal subject, such as humans, horses, and dogs. The present invention is particularly useful in treating humans.

The following examples further illustrate the present invention. These examples are intended merely to be illustrative of the present invention and are not to be construed as being limiting.

### EXAMPLES

### Example 1. In Vivo Study of Orally-Administered Orelabrutinib in Multiple Sclerosis Mouse Model

Multiple sclerosis (MS) is a chronic neuroinflammatory demyelinating disorder of the central nervous system (CNS). EAE (Experimental Autoimmune Encephalomyelitis) is the most common animal model of human MS as it is especially useful to investigate neuroinflammatory pathways. The synthesized peptide MOG₃₅₋₅₅-induced EA E mouse model was used to evaluate the therapeutic potential of orelabrutinib.

100 μL emulsion consisted of 0.75 mg/ml MOG35-55 and 4 mg/mL CFA were injected subcutaneously into two different sites on each hind flank of each C57BL/6 mouse. 300 ng of pertussis toxin in 100 μL of PBS were injected intraperitoneally at the day of immunization and two days later. Mice were randomized into 2 groups as follows: vehicle and orelabrutinib 30 mg/kg/day. Each group had 10 animals. On the day of immunization, animals were dosed QD by PO gavage with either vehicle or test compound for 21 days.

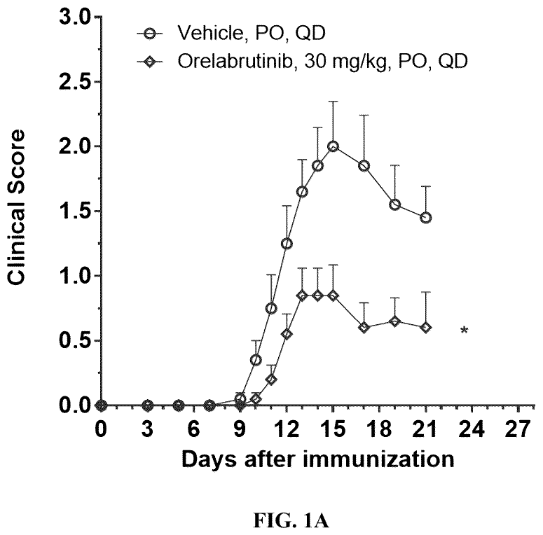

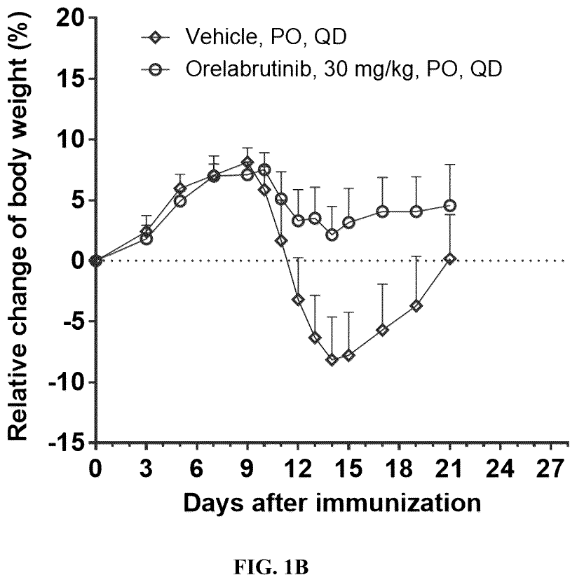

MOG₃₅₋₅₅ peptide induced mice EA E at day 9 after immunization were evaluated. EAE disease severity in control vehicle-treatment mice continued to increase from 0.4 to 2.0, reaching a peak of 2.0 at day 15 following slowly decreasing to 1.5 at day 21. The degree of body weight loss was correlated inversely with the clinical score. The body weight lost up to 10% at day 15 post-immunization. In contrast, oral orelabrutinib at 30 mg/kg/day markedly ameliorated EAE severity with reducing clinical score and body weight loss. The average score of orelabrutinib-treated mice at day 15 was 0.9 and then continued dropping to 0.6 by day 21 (p<0.05). Meanwhile, orelabrutinib reduced weight loss associated with EAE (FIGS. 1A, 1B).

In summary, oral dose of orelabrutinib significantly ameliorated EA E severity with reducing EAE score by 50% and body weight loss at 30 mg/kg/day. Orelabrutinib was efficacious to halt mouse EA E disease progression which might provide support for the development of orelabrutinib for the treatment of human MS.

### Example 2. Clinical Study of Orally Administered Orelabrutinib in Multiple Sclerosis Patients

This is a randomized, double-blind, placebo-controlled, phase 2 study of orelabrutinib in patients with relapsing-remitting multiple sclerosis (RRMS) that consists of 2 parts: a Core part and an Open-label Extension (OLE) part. The Core part is completed, and the OLE part is on-going.

The completed Core part consisted of a 4-week screening period and 24-week treatment period. Patients with RRMS were randomly assigned to 1 of 4 treatment groups in a 1:1:1:1 ratio: placebo, orelabrutinib 50 mg once daily (QD), orelabrutinib 50 mg twice daily (BID), or orelabrutinib 80 mg QD. At Week 13, patients in the placebo cohort were switched to orelabrutinib 50 mg QD.

The on-going OLE part is a single treatment arm study to enroll patients who have completed the Week 24 visit in the Core part for continued treatment and to collect additional long-term safety and efficacy data. The OLE part consists of an Entry Visit of 1 day (the day of the Week 24 visit in the Core part) and a treatment period of up to 96 weeks. After completion of the OLE or early discontinuation, patients will have a safety follow-up at approximately 4 weeks after their final dose during the OLE. In the OLE part, eligible patients receive 50 mg QD of orelabrutinib.

### Main Criteria for Inclusion:

1) Male and female subjects between 18 and 55 years old, inclusive.
2) Diagnosis of RRMS in accordance with 2017 Revised McDonald criteria (Thompson et al., 2018).
3) Neurologic stability for ≥30 days prior to both Screening and Baseline.
4) One or more documented relapses within the 2 years before Screening with either:
   a. one relapse which occurred within the last year prior to randomization, or
   b. the presence of at least 1 Gd+T1 lesion on MRI within 6 months prior to randomization
5) Have an EDSS score of 0 to 5.5 at Screening and Baseline (Day 1)
   a. Participants with an EDSS score≤2 at Screening and Baseline (Day 1) are only eligible for participation if their disease duration (time since onset of symptoms) is no more than 10 years
6) Women of childbearing potential must use a supplementary barrier method together with a highly effective method of contraception (according to ICH guidance M 3[R2]) for 4 weeks prior to randomization, throughout the trial, and for 90 days after the last dose of investigational medicinal product (IM P).

### Main Criteria for Exclusion:

Participants are excluded from the study if any of the following criteria apply:

### Medical Conditions

1) Participants diagnosed with progressive MS, in accordance with the 2017 Revised McDonald criteria including either primary progressive MS or secondary progressive MS.
2) Disease duration >10 years in participants with an EDSS≤2.0 at Screening and Baseline (Day 1).
3) Immunologic disorder other than MS or any other condition requiring oral, intravenous (IV), intramuscular, or intra-articular corticosteroid therapy, with the exception of well controlled Type 2 diabetes mellitus or well controlled thyroid disease.
4) History or current diagnosis of other neurological disorders that may mimic MS, including but not limited to: neuromyelitis optica, transverse myelitis, bilateral optic neuritis of simultaneous onset, Lyme disease, neurosarcoidosis, cerebrovascular disorders.
5) History or current diagnosis of progressive multifocal leukoencephalopathy (PML).
6) Active, clinically significant viral, bacterial, or fungal infection, or any major episode of infection requiring hospitalization or treatment with parenteral anti-infectives within 4 weeks of Screening, or completion of oral anti-infectives within 2 weeks before or during Screening, or a history of recurrent infections (i.e., 3 or more of the same type of infection in a 12-month rolling period). Vaginal candidiasis, onychomycosis, and genital or oral herpes simplex virus considered by the Investigator to be sufficiently controlled would not be exclusionary.
7) The subject
   Has a history or current diagnosis of active tuberculosis (TB), or
   Is currently undergoing treatment for latent TB infection (LTBI), or
   Has an untreated LTBI as determined by documented results within 3 months of the Screening visit of a positive TB skin test with purified protein derivative with induration ≥5 mm, or
   Has a positive QuantiFERON®-TB test at Screening

Subjects with documented completed appropriate LTBI treatment would not be excluded and are not required to be tested

Indeterminate QuantiFERON®-TB tests may be repeated once and are considered positive if retest results are positive or indeterminate

Subjects with current household contacts with active TB are also excluded
8) Individuals with a diagnosis of Gilbert's disease or chronic liver disease are excluded from the study.
9) Individuals with hemophilia, any history of serious or life-threatening bleeding events, and/or any other coagulopathy are excluded from the study.
10) History of splenectomy at any time, or any major surgery within 2 months prior to Screening.
11) History of myocardial infarction or cerebrovascular event within 6 months prior to Screening, or current active angina pectoris, history of or current congestive heart failure New York Heart Association (NY HA) Class III or Class IV, uncontrolled seizures, prolonged untreated hypertension (systolic ≥160 mm Hg and/or diastolic ≥100 mm Hg), or any other significant active medical condition in the Investigator's opinion or Sponsor's/designee's opinion.
12) A history of attempted suicide within 6 months prior to Screening or a positive response to items 4 or 5 of the Columbia-Suicide Severity Rating Scale (C-SSRS) at Screening.
13) An episode of major depression within the last 6 months prior to Screening (clinically stable minor depression is not exclusionary).
14) History of cancer, except adequately treated basal cell or squamous cell carcinoma of the skin (no more than 3 lesions requiring treatment in lifetime) or carcinoma in situ/cervical intraepithelial neoplasia of the uterine cervix, unless considered cured >5 years
15) Breastfeeding/lactating or pregnant women
16) On Screening electrocardiogram (ECG), any abnormality that in the Investigator's opinion may impact participation in the study.
17) Any other clinically significant abnormality per Investigator opinion.

### Prior/Concomitant Therapy

18) Use of injectable (e.g., IV, intramuscular, intra-articular) or oral glucocorticoids, or ACTH (e.g., A cthar gel) within 4 weeks prior to randomization. Inhaled and topical corticosteroids are allowed.
19) Treatment with beta-interferons or glatiramer acetate within 4 weeks prior to randomization.
20) Treatment with dimethyl fumarate, diroximel fumarate, or other fumaric acid esters within 4 weeks prior to randomization.
21) Treatment with teriflunomide within 4 weeks if an accelerated elimination procedure is performed or 14 weeks without the completion of an Accelerated Elimination Procedure (AEP) prior to randomization.
22) Use of natalizumab within 3 months prior to randomization. Please note that patients who have used natalizumab within 6 months prior to randomization should have an evaluation for progressive multifocal leukoencephalopathy (PML) by MRI prior to Screening
23) Use of S1P agents (e.g., fingolimod, siponimod, ozanimod, or ponesimod) within 24 weeks prior to randomization.
24) Use of IV immunoglobulin (Ig) or plasmapheresis within 12 weeks prior to randomization.
25) Treatment at any time with rituximab, ofatumumab, or ocrelizumab, except:
   Participants who have received only a single dose of rituximab, ofatumumab, or ocrelizumab, and reason for treatment discontinuation was not treatment failure, and the last dose was at least 48 weeks prior to randomization.
26) Treatment at any time with daclizumab, any B cell depleting therapy, BTK inhibitors, mitoxantrone, or lymphocyte-depleting therapies (e.g., alemtuzumab, anti-CD4, cladribine, cyclophosphamide, azathioprine, total body irradiation, bone marrow transplantation, or other such therapies not specifically listed).
27) Concomitant treatment with dalfampridine (Ampyra®) or fampridine can be included only if they have been on a stable dose for 3 months prior to randomization.
28) Treatment with medical marijuana for MS symptoms are excluded, unless it is consistent with local MS treatment guidelines and local regulations. Non-THC containing cannabinoids are allowed. Dosage, formulation, and route of administration should be recorded as a concomitant medication.
29) On anticoagulation, or antiplatelet therapy, except:
   Low dose (81 mg) daily aspirin for cardioprotection.
   Fish oil supplements stopped 4 weeks prior to randomization.
30) NSAIDs used regularly instead of being on an as-needed basis. Regular use of aspirin for cardioprotection purpose is acceptable (see #29).
31) Use of strong to moderate inducers of CYP3A within 3 weeks prior to treatment, or use of strong to moderate inhibitors of CYP3A within 1 week prior to treatment, or possible concurrent use of strong to moderate inducer or inhibitor of CYP3A with the study drug during the treatment period.

### Prior/Concurrent Clinical Study Experience

32) Participation in any investigational drug study within 6 months or 5 half-lives of the investigational drug, whichever is longest, prior to Screening.

### Diagnostic Assessments

33) Any of the following:
   a. History of or positive for human immunodeficiency virus (HIV) at Screening.
   b. History of or positive for hepatitis C virus (HCV) antibody at Screening.
   C. Positive for hepatitis B surface antigen (HBsAg) and/or positive for hepatitis B core antibody at Screening.
34) Estimated glomerular filtration rate (eGFR)<30 mL/min/1.73 m² as calculated by the 4 variable Modification of Diet in RenalDisease equation by the central laboratory or any renal condition that would preclude the administration of gadolinium (e.g., acute kidney injury).
35) ALT, AST, amylase, or lipase >2×ULN of laboratory reference range, total bilirubin >1.5×ULN, or any other clinically significant laboratory abnormality.
36) Significant cytopenia, including neutrophil count <1,500/mm³, platelet count <75,000/mm³, absolute lymphocyte count <1,000/mm³, or a white blood cell count <3,500/mm³.
37) International normalized ratio (INR)≥1.5 or activated partial thromboplastin time (APTT)≥1.5×ULN
38) B cell CD19 count below the lower limit of normal at Screening

### Other Exclusions

39) Any allergy, contraindication, or inability to tolerate orelabrutinib or any of the excipients in the Study intervention (e.g., orelabrutinib tablets, placebo tablets).
40) Inability to comply with MRI scanning, including contraindications to MRI such as known allergy or other contraindications to gadolinium contrast media, claustrophobia, presence of a pacemaker, cochlear implants, ferromagnetic devices or clips, intracranial vascular clips, insulin pumps, nerve stimulators.
41) Vaccination with live or live-attenuated virus vaccine within 1 month prior to Screening.
42) Regular alcohol consumption within 6 months prior to the study defined as: an average weekly intake of >14 units for males or >7 units for females. One unit is equivalent to 8 g of alcohol: a half pint (˜240 mL) of beer, 1 glass (125 ml) of wine or 1 (25 mL) measure of spirits.

### Dose and Mode of Administration:

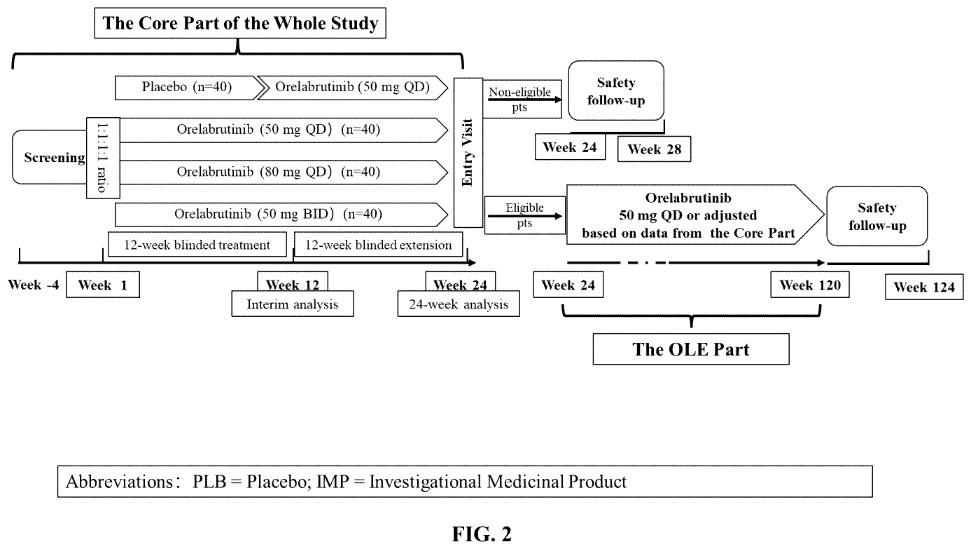

The study design of Example 2 is shown in FIG. 2. Patients with RRMS were randomly assigned to 1 of 4 treatment groups: placebo, orelabrutinib (50 mg, once daily [QD]), orelabrutinib (80 mg QD) and orelabrutinib (50 mg, twice daily [BID]) at a 1:1:1:1 ratio.
Cohort 1: Placebo (Week 1-12)/orelabrutinib 50 mg QD (Week 13-24)
Cohort 2: Orelabrutinib 50 mg QD (Week 1-24)
Cohort 3: Orelabrutinib 80 mg QD (Week 1-24)
Cohort 4: Orelabrutinib 50 mg BID (Week 1-24)

At week 12 of the treatment period, subjects in Cohort 1 were switched to orelabrutinib at a starting daily dose of 50 mg. A separate open-label extension (OLE) study was developed to allow continued dosing, provided that safety and tolerability were acceptable.

Placebo or orelabrutinib was orally administered daily starting on Day 1 of the treatment period. Subjects returned to study site on Day 8 and every 4 weeks starting from Day 1 for trial visits and were assessed for safety and efficacy.

### Clinical Trial Duration:

The study consisted of a Screening period of up to 4 weeks, a Treatment period of 24 weeks, and a Safety Follow-Up period of 4 weeks.

### Primary Objective:

To evaluate the efficacy of orelabrutinib on the cumulative number of new gadolinium-enhancing (GdE) T1 magnetic resonance (MRI) brain lesions versus placebo over 12-24 weeks of treatment.

### Primary Endpoints:

Cumulative number of new Gd+T1 brain lesions up to Week 12 compared to placebo.

### Secondary Objectives:

To evaluate the efficacy of orelabrutinib on clinical symptoms and imaging measures versus placebo.

To evaluate the safety and tolerability of orelabrutinib.

### Secondary Endpoints:

Evaluate the efficacy and safety of orelabrutinib compared to placebo
Cumulative number of new Gd+T1 brain lesions up to Week 24 compared to placebo.
Total number of GdE T1 lesions at Weeks 12, 16, 20 and 24.
Total number of new or enlarging T2 lesions at Weeks 12, 16, 20 and 24.
Annualized relapse rate (ARR), based on protocol-defined qualified relapses, at Weeks 12 and 24.
Proportion of subjects who remain qualified relapse-free at Weeks 12 and 24.
Change from baseline in EDSS at Weeks 12 and 24.
Change from baseline in the volume of GdE T1 lesions at Weeks 12 and 24.
Change from baseline in the volume of T2. lesions at Weeks 12 and 24.
Safety as assessed by the nature, severity, and incidence of adverse events (AEs) (graded according to National Cancer Institute-common Terminology Criteria for AEs, NCI-CTCAE version 5.0); vital signs; electrocardiograms (ECGs); and clinical laboratory safety parameters.

### Summary of Results

### Study Population

Intent-to treat (ITT) population. 158 patients randomized: 40 in placebo, 37 in 50 mg QD cohort, 41 in 50 mg BID cohort, and 40 in 80 mg QD cohort.
3 force majeure events during the study caused missing MRI data at Week 4, 8, or 12 timepoints: Ukraine war & crisis, COVID-19 pandemic outbreak, and FDA's partial clinical hold.
Post-hoc analysis set (PHS) population. 115 patients remaining after excluding patients who missed any one of the 3 MRI data points within first 12 weeks: 27 in placebo, 30 in 50 mg QD, 29 in 50 mg BID, and 29 in 80 mg QD.
132 patients completed 12 weeks of treatment and 127 patients completed 24 weeks of treatment.

### Primary Endpoint

### Cumulative Number of New Gd+T1 Brain Lesions Up to Week 12

In the ITT (intent-to-treat) population, percent lesion reductions at Week 12 relative to placebo for orelabrutinib treatment cohorts of 50 mg QD, 50 mg BID, and 80 mg QD were 73.3%, 78.8%, and 80.5%, respectively, and all reached statistical significance.

In the PHS (post-hoc analysis) population, percent lesion reductions at Week 12 relative to placebo for orelabrutinib treatment cohorts of 50 mg QD, 50 mg BID, and 80 mg QD were 73.8%, 81.0%, and 90.4%, respectively, and all reached statistical significance.

Table 1 shows cumulative number of new Gd+T1 brain lesions at Week 12 by analysis population. (PHS Population, N=115) The adjusted mean cumulative number, percent reduction (orelabrutinib vs placebo) associated with p-value are estimated from a Poisson regression model with a Pearson scale parameter with a log link function and offset by log number of scans as of Week 12. Baseline number of Gd+T1 brain lesions is included in the model as a continuous covariate.

TABLE 1

| | Placebo/ | | | |
|---|---|---|---|---|
| | Orela | Orela | Orela | Orela |
| | 50 mg QD | 50 mg QD | 50 mg BID | 80 mg QD |
| | (N = 27) | (N = 30) | (N = 29) | (N = 29) |
| Adjusted mean | 4.34 | 1.14 | 0.82 | 0.42 |
| cumulative number | | | | |
| of new Gd + T1 | | | | |
| lesions | | | | |
| at Week 12 | | | | |
| % Reduction | | 73.8 | 81.0 | 90.4 |
| P value | | 0.0278 | 0.0073 | 0.0018 |

The primary endpoint was achieved for all active treatment cohorts, with the 80 mg QD cohort showing the highest reduction of 90.4% in new Gd+T1 brain lesions at Week 12.

### Main Secondary Endpoints

### Cumulative Number of New Gd+T1 Lesions Up to Week 24

Control of new T1 lesions was achieved in all orelabrutinib cohorts after 4 weeks of treatment, with the effect sustained up to 24 weeks. In the PHS population, all three original active treatment cohorts of 50 mg QD, 50 mg BID, and 80 mg QD still showed significant cumulative lesion reductions of 67.4%, 83.3%, and 92.3%, respectively, from Week 4 throughout Week 24 compared to placebo, despite the placebo arm switching to 50 mg QD treatment after Week 12. The 80 mg QD dose remained the best dose for lesion control throughout 24 weeks.

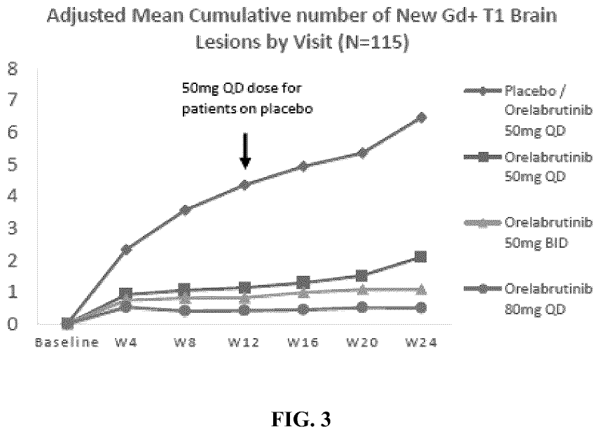

All orelabrutinib cohorts achieved control of new T1 lesions after 4 weeks of treatment, and the effect was sustained up to 24 weeks. Orelabrutinib at 80 mg QD remained the best dose level for T1 lesion control throughout 24 weeks. Despite the placebo arm switching to 50 mg QD treatment after Week 12, all three original active treatment cohorts still showed significant cumulative lesion reductions from Week 4 throughout Week 24 compared to the placebo/50 mg QD switch arm (FIG. 3 and Table 1).

FIG. 3 shows adjusted mean cumulative number of new Gd+T1 brain lesions by visit up to Week 24 (PHS, N=115). The adjusted mean cumulative numbers are estimated from Poisson regression models with a Pearson scale parameter with a log link function and offset by log number of scans as of that visit. Baseline number of Gd+T1 brain lesions is included in the model as a continuous covariate.

Table 2 shows cumulative number of new Gd+T1 lesions from Week 4 to 24. (PHS Population, N=115).

TABLE 2

| | Placebo/ | | | |
|---|---|---|---|---|
| | Orela | Orela | Orela | Orela |
| | 50 mg QD | 50 mg QD | 50 mg BID | 80 mg QD |
| | (N = 27) | (N = 30) | (N = 29) | (N = 29) |
| Adjusted mean | 6.45 | 2.10 | 1.08 | 0.50 |
| cumulative number | | | | |
| of new Gd + T1 | | | | |
| lesions | | | | |
| from W 4 to W 24 | | | | |
| % Reduction | | 67.4 | 83.3 | 92.3 |
| P value | | 0.0958 | 0.0114 | 0.0037 |

### Annualized Relapse Rate (ARR) Up to Week 24

The ARRs were reduced for all orelabrutinib cohorts compared to the placebo group at Week 12, with sustained reductions through Week 24. In the PHS population, low ARRs of 0.05, 0.12, and 0.06 were achieved at Week 24 in the 50 mg QD, 50 mg BID, and 80 mg QD cohorts, respectively, compared to 0.14 in the placebo/50 mg QD switch arm.

### Efficacy Conclusion

There were statistically significant reductions in the cumulative number of new Gd+T1 lesions at Week 12 in all orelabrutinib treatment cohorts of 50 mg QD, 50 mg BID, and 80 mg QD compared to placebo. The 80 mg QD cohort showed the highest reduction of 90.4%. Efficacy appears to be dose-dependent increasing in the order of 50 mg QD, 50 mg BID, and 80 mg QD, suggesting a potential Cmax-driven efficacy correlation.

Control of new T1 lesions was sustained up to 24 weeks in all original orelabrutinib treated cohorts. Orelabrutinib at 80 mg QD remained the best dose level for Gd+T1 lesion control throughout 24 weeks, with the highest lesion reduction of 92.3% at Week 24. Despite the placebo arm switching to 50 mg QD treatment after Week 12, all 3 original active treatment groups still showed significant cumulative lesion reductions from Week 4 throughout Week 24 compared to the placebo/50 mg QD arm. Moreover, the annual relapse rates of all active treatment groups were reduced at 24 weeks.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method for treating multiple sclerosis (MS), comprising the step of orally administering to a subject in need thereof 50-100 mg/day of orelabrutinib for at least a month.

2. The method of claim 1, wherein the subject is treated with 50-80 mg/day of orelabrutinib.

3. The method of claim 1, wherein the subject is treated with 50-80 mg of orelabrutinib once daily.

4. The method of claim 1, wherein the subject is treated with about 80 mg of orelabrutinib once daily.

5. The method of claim 1, wherein the subject is treated for 3 months to 2 years.

6. The method of claim 2, wherein the subject is treated for 3 months to 2 years.

7. The method of claim 3, wherein the subject is treated for 3 months to 2 years.

8. The method of claim 4, wherein the subject is treated for 3 months to 2 years.

9. The method of claim 1, wherein the method treats relapsing-remitting MS, secondary progressive MS, primary progressive MS, and progressive relapsing MS.

10. The method of claim 9, wherein the method treats relapsing-remitting MS.
